(19) United States
(12) Patent Application Publication
(10) Pub. No.: US 2004/0192756 A1
(43) Pub. Date: Sep. 30, 2004

(54) Amino-acid-based compositions, suitable in therapy for the healing and/or mending of wounds and lesions, in particular for application in the opthalmic field

(76) Inventors: Franco Conti, Via della Spiga (IT); Francesco Saverio Dioguardi, Milano (IT)

Correspondence Address: Sughrue Mion, 2100 Pennsylvania Avenue NW, Washington, DC, 20037-3202, US

(21) Appl. No.: 10/486,141

(22) PCT Filed: Jun. 10, 2002
(86) PCT No.: PCT/IB02/02147

(30) Foreign Application Priority Data
Aug. 8, 2001 (IT) TO2001A000804

(51) Int. Cl. A61K 31/405; A61K 31/401; A61K 31/198

(52) U.S. Cl. 514/419; 514/423; 514/561; 514/564; 514/567

(57) ABSTRACT

Amino-acid-based compositions, suitable in therapy for the healing of wounds and lesions, in particular for application in the ophthalmic field, comprising: proline, glycine and lysine, up to 80 wt % on the total of all the amino acids or active ingredients envisaged; one or more of the amino acids selected in the group comprising leucine, isoleucine and threonine, in an overall quantity of between 2 wt % and 60 wt % on the total of all the amino acids or active ingredients envisaged. Preferably envisaged are also other essential amino acids, in particular methionine, phenyl alanine, histidine, tryptophan, and non-essential amino acids, in particular tyrosine and cyst(e)ine (i.e., cystine and cysteine). Other amino acids can be added, provided that their sum is a percentage lower than 20 wt % with respect to the sum of the other active ingredients, and less than 10 wt % for each individual amino acid.

The present invention relates to amino-acid-based compositions, suitable in therapy for the healing and/or mending of wounds and lesions, in particular for application in the ophthalmic field.

From U.S. Pat. No. 5,198,465 a composition is known, with a base of proline, glycine and lysine, possibly comprising also methionine, cystine, cysteine, α-ketoglutaric acid and vitamin C, the said composition being able to induce or promote biological synthesis of collagen in situations in which said synthesis is lacking.

Starting from the above known technique, the purpose of the present invention is to indicate new amino-acid-based compositions which will prove particularly effective in therapy for healing and/or mending wounds and lesions, in particular for application in the ophthalmic field.

In this perspective, the inventors have succeeded in obtaining the formulation of amino-acid-based compositions in accordance with the attached claims, which are intended as forming an integral part of the present description, that prove particularly effective in view of the purposes proposed.

The above compositions comprise, as main active ingredients glycine, proline and lysine, the sum of which is up to 80 wt % on the total of all the amino acids or active ingredients envisaged.

The compositions according to the invention are then characterized in that they envisage, as further active ingredients, one or more amino acids selected in the group comprising leucine, isoleucine and threonine in an overall quantity of between 2 wt % and 60 wt % on the total of all the amino acids or active ingredients envisaged.

Preferably, the compositions comprise, as further active ingredient, valine; in this case, the sum in weight of leucine, isoleucine, valine and threonine is preferably up to 75 wt % on the total of all the amino acids or active ingredients envisaged.

The compositions may possibly envisage, as further active ingredients, other essential amino acids, in particular phenyl alanine and/or histidine and/or tryptophan and/or methionine, and non-essential amino acids, in particular tyrosine and/or cyst(e)ine (i.e., cystine and cysteine).

Preferably the sum of the amounts expressed as molecular weight of threonine and lysine is greater than the sum of the individual quantities of the other essential amino acids present, but in any case smaller than either the sum of the individual amounts of glycine and proline or than the sum of the individual amounts of leucine, isoleucine and valine.

In addition, the amounts expressed in molecular weights of threonine and lysine may each be greater than the individual amounts of the other essential amino acids envisaged, but, preferably, the amount of threonine is smaller than the individual amounts of glycine, proline, leucine, isoleucine and valine, and/or the amount of lysine is smaller than the individual amounts of glycine, proline and leucine, and/or the amount of threonine is smaller than the amount of lysine.

The compositions according to the invention may moreover comprise one or more additional amino acids, with respect to the ones mentioned previously, the sum of which, expressed in molecular weights, is preferably of a percentage smaller than 20% with respect to the sum of the other active ingredients, and less than 10% for each individual additional amino acid.

The preferred formulations of the compositions according to the invention, comprising essential and non-essential amino acids (glycine, proline, lysine, leucine, isoleucine, valine, threonine, methionine, phenyl alanine, histidine, tryptophan, tyrosine and cyst(e)ine) fall within the following spheres (in what follows, where not otherwise specified, the weight percentages of the various amino acids on the total thereof are indicated):

glycine (8-40 wt %), proline (7-40 wt %), lysine (3-35 wt %), which account for 18-80 wt % of the entire composition of amino acids;

leucine (4-40 wt %), isoleucine (2-20 wt %), valine (2-20 wt %), threonine (up to 20 wt %), which account for 8-70 wt % of the entire composition of amino acids, where leucine, isoleucine and valine are preferably in a stoichiometric ratio 2:1:1 and where threonine plus lysine are preferably in a molar ratio with respect to one another with leucine, isoleucine and valine of between 20 and 70%, preferably with a ratio between threonine and lysine in which lysine is more represented than threonine; and

histidine, present in molar fractions of up to 50% of the following amino acids:

cyst(e)ine (i.e., cystine and cysteine) and methionine, up to 50% of the histidine, where the ratio between cyst(e)ine and methionine should preferably be between 50 and 200% greater than the cyst(e)ine, in molar ratio;

phenyl alanine and tyrosine, in a molar ratio of up to 60% of the histidine (where the tyrosine is preferably represented by up to 50% of the molar weight of the phenyl alanine);

tryptophan up to 5% of the weight of all the other amino acids on a basis of molar weight.

As has been said, any other amino acid can be added to the aforesaid formulation without altering the expected effects thereof, provided that the sum of the additional amino acids is in a percentage lower than 20 wt % with respect to the sum of the other active ingredients (less than 10 wt % for each amino acid).

With the aim of demonstrating the effectiveness of the mixture according to the invention for the purposes of therapy for healing wounds and lesions, experimental tests have been carried out, aimed at testing the stimulating action on the cell-proliferation activity of two stocks of cells (corneal fibroblasts and conjunctival cells) performed by two mixtures of amino acids.

The first mixture was obtained according to the teachings of U.S. Pat. No. 5,198,465 and contained only glycine, proline, lysine and vitamin C.

The second mixture, obtained according to the present invention, had the following composition:

| | Amounts in mg | Weight percent |
|---|---|---|
| | (per g. of | (on the total of |
| Amino acids | mixture) | amino acids) |
| Glycine | 250.0 | 25.00% |
| Proline | 218.8 | 21.88% |
| Lysine | 112.5 | 11.25% |
| Leucine | 156.3 | 15.63% |
| Isoleucine | 78.1 | 7.81% |
| Valine | 78.1 | 7.81% |
| Threonine | 43.8 | 4.38% |
| Methionine | 6.3 | 0.63% |
| Phenyl alanine | 12.5 | 1.25% |
| Histidine | 18.8 | 1.88% |
| Tryptophan | 2.5 | 0.25% |
| Tyrosine | 3.8 | 0.38% |
| Cyst(e)ine | 18.8 | 1.88% |

The activity of the two mixtures subjected to comparative analysis was tested both in vitro and in vivo.

In-Vitro Tests

The cell lines chosen for development of the experimental model were: rabbit corneal fibroblast cells (SIRC) and human conjunctival cells (1-5C-4). The cell lines used were exposed to a dose-response curve developed in a concentration range of from 0.1 mg/ml to 1 mg/ml of the two mixtures of amino acids, i.e., the mixture obtained according to the teachings of U.S. Pat. No. 5,198,465 and the mixture obtained in accordance with the present invention.

The products were solubilized and then diluted at the experimental concentrations, using a culture medium without any serum component. The cell response to exposure was assessed using the MTT calorimetric test, a method which enables definition of the residual vitality of the cells exposed to the product, quantifying the metabolic functionality of the mitochondria. This evaluation was made on the 3^{rd}, 6^{th} and 8^{th} day.

For each experimental point of the dose-response curve, the cell response of 8 wells was evaluated. The values of absorbance were subjected to statistical analysis for determination of the mean value, the standard error and significance (Student's t).

In the tables given below, which summarize the data obtained from the individual recordings, the numerical values are expressed as percentage of cell vitality with respect to a control that had not been exposed to the product, and to which a vitality of 100% was attributed.

Tables 1 and 2 appearing below show, in particular, the dose-response curve in the absence of bovine foetal serum of fibroblasts, respectively for the mixture obtained according to the teachings of U.S. Pat. No. 5 198 465 and for the mixture according to the invention.

TABLE 1
(mixture according to US-A-5,198,465)

| mg/ml | 3^{rd} day | 6^{th} day | 8^{th} day |
|---|---|---|---|
| 0 | 100 | 100 | 100 |
| 0.1 | 126.02 * | 131.08 # | 206.6 # |
| 0.25 | 129.27 * | 148.09 # | 219.4 # |
| 0.5 | 127.78 * | 169.65 # | 235.41 # |
| 1 | 125 * | 153.75 # | 229 # |

* = p < 0.005 vs. control
# = p < 0.0001 vs. control

TABLE 2
(mixture according to the invention)

| mg/ml | 3^{rd} day | 6^{th} day | 8^{th} day |
|---|---|---|---|
| 0 | 100 | 100 | 100 |
| 0.1 | 140.31 #° | 178.70 #°° | 220.53 #° |
| 0.25 | 154.01 #° | 180.42 #°° | 230.91 #° |
| 0.5 | 160.22 #° | 193.08 #°° | 260.07 #° |
| 1 | 163.04 #° | 190.23 #°° | 261.18 #°° |

# = p < 0.0001 vs. control
° = p < 0.005 vs. US-A-5,198,465 mixture
°° = p < 0.0001 vs. US-A-5,198,465 mixture

As may be seen from Table 1, the mixture according to the known art expresses a stimulant activity on the proliferation of the fibroblasts that is already statistically significant on the third day for all the concentrations tested and becomes more evident on the sixth day and on the eighth day with increments that are approximately dose-dependent. From Table 2, it may be noted, instead, how the mixture according to the invention is able to produce a faster stimulation in time, with increments in comparison with the mixture according to the prior art that are already statistically significant on the third day. From a comparison between Tables 1 and 2, it clearly emerges how the mixture according to the invention is decidedly more effective on the proliferation of fibroblasts, which thus leads to a reduction in the response times and to a further increase in the number of cells.

Tables 3 and 4 appearing below show, instead, the dose-response curve in the absence of bovine foetal serum in the conjunctival-cell line, respectively for the mixture obtained according to the teachings of U.S. Pat. No. 5,198,465 and for the mixture according to the invention.

TABLE 3
(mixture according to US-A-5,198,465)

| mg/ml | 3^{rd} day | 6^{th} day | 8^{th} day |
|---|---|---|---|
| 0 | 100 | 100 | 100 |
| 0.1 | 91.14 | 102.32 | 109.71 |
| 0.25 | 104.3 | 107.91 | 108.08 |
| 0.5 | 111.06 | 109.84 | 110.13 |
| 1 | 101.02 | 113.06 * | 118.77 * |

* = p < 0.005 vs. control

TABLE 4
(mixture according to the invention)

| mg/ml | 3^{rd} day | 6^{th} day | 8^{th} day |
|---|---|---|---|
| 0 | 100 | 100 | 100 |
| 0.1 | 87.7 | 108.16 *° | 138.97 #°° |
| 0.25 | 112.83 | 122.45 *° | 178.07 #°° |
| 0.5 | 100.97 | 133.13 *° | 193.81 #°° |
| 1 | 113.91 | 126.26 *°° | 201.68 #°° |

* = p < 0.005 vs. control
# = p < 0.0001 vs. control
° = p < 0.005 vs. US-A-5,198,465 mixture
°° = p < 0.0001 vs. US-A-5,198,465 mixture

From Table 3, it may be noted how the conjunctival cells presented a poor response to the addition of the mixture according to the prior art in the culture medium, and only at the dose of 1 mg/ml at day 6 and day 8 showed a modest significant increase.

From Table 4, it may instead be noted how the mixture according to the invention did not reveal a significant increase at day 3, but at day 6 and day 8 the increments in cell proliferation were markedly evident and significant both in regard to the controls and in regard to the mixture according to the prior art.

From the above results, it is thus evident how the mixture according to the invention is able to stimulate the two fundamental cell stocks for repairing corneal lesions, ensuring a rapid formation of the corneal stroma, of the basal lamina, and hence a fast re-epithelization of the mucosae.

In-Vivo Tests

To test the effectiveness of the mixture of amino acids according to the invention as compared to the mixture according to U.S. Pat. No. 5,198,465, 20 patients affected by corneal ulcers for over 18 months and resistant to normal treatments were chosen, who had been observed for three months prior to start of treatment.

The group consisted of 12 men and 8 women with an average age of 58 years. Of these 6 were diabetics of type 2, who were being treated with drugs of a hypoglycaemic type and were in good metabolic compensation.

The cases were randomly divided into two groups of 10 patients each, with 3 diabetics in each group.

Throughout the experimental period, no drug was changed, either topical drug or drug to be administered via general route, and only the two mixtures according to the invention were added. The mixture according to the prior art was administered via oral route at the dosage of 12 g divided into three administrations per day. Also the mixture according to the invention was administered via oral route at the dosage of 12 g, once again divided into three administrations per day. Treatment lasted one month.

The evaluation of the therapeutic activity envisaged just two possibilities: complete healing of the lesion; and no healing within one month of treatment.

Of the 10 subjects treated with the mixture according to the invention, complete healing was found in 9 subjects, whereas using the mixture according to U.S. Pat. No. 5,198,465, a case of healing was found in just one subject and improvements in a further four subjects (reduction in the diameter of the lesion).

From the above findings, it is thus noted how the treatment with the mixture according to the invention has proven itself to be clearly superior to treatment with the mixture according to U.S. Pat. No. 5,198,465, with a healing of the lesion in 90% of the cases (p<0.05).

The compositions according to the invention may be employed for administration via oral route (pills, tablets, powders, etc.), for topical administration (collyrium, cream, gel, etc.), and for administration via parenteral route, for example via local injection. In connection with this latter possibility of use, an injectable aqueous solution may be envisaged, prepared extemporarily, dissolving the composition according to the invention, prepared previously in a lyophilized form, in a biologically compatible aqueous liquid (distilled water, physiological solution or other aqueous solution).

If so required, administration of the mixture may be in the form of a number of distinct preparations, for instance a tablet (or any other pharmaceutical formulation) containing some of the amino acids envisaged and/or fractions thereof (for example, glycine, proline, lysine), and a tablet (or any other pharmaceutical formulation) containing the other amino acids envisaged and/or fractions thereof (for example, leucine, isoleucine, threonine, and possibly lysine and/or methionine and/or phenyl alanine and/or histidine and/or tryptophan and/or tyrosine and/or cyst(e)ine).

Of course, for the purposes of preparation of the compositions according to the invention, it is possible to use diluents and excipients in any pharmacological form suited for the chosen use.

From the foregoing description, there emerge clearly the characteristics of the present invention, as likewise do the advantages afforded thereby, which are chiefly represented by the considerable efficacy in the therapy of healing and/or mending of wounds, lesions and ulcers, in particular by means of the increased proliferation of the cells of the stroma. The mixtures according to the present invention prove highly effective in the treatment of corneal ulcers and in the field of refractive surgery, but the sphere of application of the invention must not be understood as being limited to the area of ophthamology. In such a perspective, the invention must therefore be understood as extending to all those applications in which it is desirable to have a rapid healing or mending of lesions of any type, including bone fractures and traumas to internal organs.

The compositions according to the present invention may possibly envisage the addition of α-ketoglutaric acid, up to 20 wt % of the total weight, and vitamin C, between 10 wt % and 50 wt % of the total weight, the latter functioning, in particular, as co-enzyme of specific hydroxylase in the catalysis of the biological synthesis of collagen.

1-22. (canceled)

23. Amino-acid-based composition, suitable in therapy for the healing and/or mending of wounds and lesions, in particular for application in the ophthalmic field, comprising:
proline, glycine and lysine, in a total amount of up to 80 wt % on the total of all the amino acids or active ingredients envisaged,
leucine, isoleucine and threonine, in an overall quantity of between 2 wt % and 60 wt % on the total of all the amino acids or active ingredients envisaged,—valine,
one or more of the other essential amino-acids phenyl alanine, histidine, tryptophan and methionine,
wherein
the amounts expressed as molecular weight of threonine and lysine are each greater than the sum of the individual amounts of the said one or more other essential amino acids present in the composition, but:
the amount of threonine is smaller than the individual amounts of glycine, proline, lysine, leucine, isoleucine and valine;
the amount of lysine is smaller than the individual amounts of glycine, proline and leucine.

24. Composition according to claim 23, wherein the sum of the amounts expressed as molecular weight of threonine and lysine
is smaller than the sum of the individual amounts of glycine and proline, and/or
is smaller than the sum of the individual amounts of leucine, isoleucine and valine.

25. Composition according to claim 23, wherein the sum of leucine, isoleucine, valine and threonine is up to 75 wt % of the total of all the amino acids or active ingredients envisaged.

26. Composition according to claim 23, comprising at least of between methionine and tyrosine as further active ingredient.

27. Composition according to claim 23, comprising cyst(e)ine, i.e., cystine and cysteine, as further active ingredient, preferably in a molar ratio with methionine equal to or higher than 2:1.

28. Composition according to claim 27, further comprising one or more additional amino acids, the sum of which, expressed in molecular weight, is in a percentage lower than 20% with respect to the sum of the other active ingredients and less than 10% for each individual additional amino acid.

29. Composition according to claim 23, comprising:
between 8 wt % and 40 wt % of glycine on the total of the amino acids envisaged;
between 7 wt % and 40 wt % of proline on the total of the amino acids envisaged;
between 3 wt % and 35 wt % of lysine on the total of the amino acids envisaged;
the sum of glycine, proline and lysine being, in particular, not less than 18 wt % of the total of the amino acids envisaged.

30. Composition according to claim 23, comprising:
between 4 wt % and 35 wt % of leucine on the total of the amino acids envisaged;
between 2 wt % and 20 wt % of isoleucine on the total of the amino acids envisaged;
between 2 wt % and 20 wt % of valine on the total of the amino acids envisaged;
up to 20 wt % of threonine on the total of the amino acids envisaged.

31. Composition according to claim 30, wherein the sum of leucine, isoleucine, valine and threonine is between 10 wt % and 50 wt % on the total of the amino acids envisaged.

32. Composition according to claim 30, wherein leucine, isoleucine and valine are in a ratio of 2:1:1.

33. Composition according to claim 23, characterized in that threonine plus lysine are in a molar ratio with respect to one another with leucine, isoleucine and valine of between 20% and 70%, preferably with a ratio between threonine and lysine in which lysine is more represented than threonine.

34. Composition according to claim 23, characterized in that histidine is present in a molar ratio of up to 50% of the following amino acids:
cyst(e)ine (i.e., cystine and cysteine) and methionine;—phenyl alanine and tyrosine;
tryptophan, in particular in a molar ratio of up to 5% of the weight of all the other amino acids on a basis of molar weight.

35. A method for the healing and/or mending of wounds and lesions in a body, comprising administering thereto
proline, glycine and lysine, up to 80 wt % on the total of all the administered amino acids,
leucine, isoleucine and threonine, in an overall quantity of between 2 wt % and 60 wt % on the total of all the administered amino acids,—valine,
one or more of the other essential amino-acids phenyl alanine, histidine, tryptophan and methionine,
wherein
the amounts, expressed as molecular weight, of threonine and lysine are each greater than the sum of the individual amounts of the said one or more other essential amino acids present in the composition, but:
the amount of threonine is smaller than the individual amounts of glycine, proline, lysine, leucine, isoleucine and valine;
the amount of lysine is smaller than the individual amounts of glycine, proline and leucine.

36. Method according to claim 35, wherein the sum of the amounts, expressed as molecular weight, of threonine and lysine
is smaller than the sum of the individual amounts of glycine and proline, and/or
is smaller than the sum of the individual amounts of leucine, isoleucine and valine.

37. Method according to claim 35, wherein at least one of methionine and tyrosine is further administered.

38. Method according to claim 35, wherein cyst(e)ine, i.e., cystine and cysteine, is further administered, preferably in a molar ratio with methionine equal to or higher than 2:1.

39. Method according to claim 38, wherein one or more additional amino acids are administered, the sum of which, expressed in molecular weight, is in a percentage lower than 20% with respect to the sum of the other amino acids and less than 10% for each individual additional amino acid.

40. Amino-acid-based composition for the healing and/or mending of wounds and lesions comprising:
proline, glycine and lysine, in a total amount of up to 80 wt % on the total of all the amino acids or active ingredients envisaged,
leucine, isoleucine and threonine, in an overall quantity of between 2 wt % and 60 wt % on the total of all the amino acids or active ingredients envisaged,—valine,
wherein the sum of the amounts expressed as molecular weight of proline, glycine, lysine and threonine is at least twice the sum of the amounts expressed as molecular weight of leucine, isoleucine and valine.

41. Composition according to claim 40, wherein the amount of threonine expressed as molecular weight is smaller than the individual amounts of glycine, proline and lysine.

42. Composition according to claim 40, wherein the amount of lysine expressed as molecular weight is smaller than the individual amounts of glycine, proline and leucine.

43. Composition according to claim 40, further comprising one or more other essential amino-acids selected in the group consisting of phenyl alanine, histidine, tryptophan and methionine.

44. Composition according to claim 43, wherein the sum of the amounts expressed as molecular weight of threonine and lysine is greater than the sum of the individual amounts of the said one or more other essential amino acids present in the composition.

45. Composition according to claim 43, wherein the amounts expressed as molecular weight of threonine and lysine are each greater than the sum of the individual amounts of the said one or more other essential amino acids present in the composition.

46. Composition according to claim 40, further comprising at least one of methionine, tyrosine and cyst(e)ine, i.e., cystine and cysteine, as further active ingredient.

47. A method for the healing and/or mending of wounds and lesions in a body, comprising administering thereto proline, glycine, lysine and threonine, wherein
the administered amounts of threonine, expressed as molecular weight, is smaller than the administered individual amounts of proline, glicine and lysine, and
the administered amounts of lysine, expressed as molecular weight, is smaller than the administered individual amounts of proline and glicine.

48. Method according to claim 47, further comprising administering at least one further amino acid selected in the group consisting of leucine, isoleucine and valine.

49. Method according to claim 48, wherein the administered amount of threonine, expressed as molecular weight, is smaller than the administered individual amounts of at least one of leucine, isoleucine and valine.

50. Method according to claim 48, wherein the administered amount of lysine, expressed as molecular weight, is smaller than the administered amount of leucine.

51. Method according to claim 47, further comprising administering one or more additional amino acid selected in the group consisting of phenyl alanine, histidine, tryptophan and methionine.

52. Method according to claim 51, wherein the sum of the administred amounts, expressed as molecular weight, of threonine and lysine is greater than the sum of the individual administered amounts of the said one or more additional amino acids.

53. Method according to claim 51, wherein the administered amounts, expressed as molecular weight, of threonine and lysine are each greater than the sum of the administered individual amounts of the said one or more additional amino.

54. Method according to claim 51, further comprising administering at least one of methionine, tyrosine and cyst(e)ine, i.e., cystine and cysteine.
